(19) United States
(12) Patent Application Publication
(10) Pub. No.: US 2011/0220123 A1
(43) Pub. Date: Sep. 15, 2011

(54) Anti-snoring device using naturally generated positive pressure

(76) Inventor: Jack D. Robson, (US)

(21) Appl. No.: 12/723,668

(22) Filed: Mar. 14, 2010

(51) Int. Cl. A61F 5/56

(52) U.S. Cl. 128/848

(57) ABSTRACT

In one embodiment, a device for reducing or eliminating snoring by providing easy inhalation through the mouth and slightly restricted exhalation allowed only through the nose. This results in positive airway pressure, keeping the soft tissues of the mouth and throat sufficiently separate to discourage snoring.

### BACKGROUND OF THE INVENTION

1. Field of Invention

The present invention relates to an anti-snoring device.

2. Discussion of the Prior Art

Snoring and obstructive sleep apnea (OSA) affects nearly 20% of all adults. Beyond causing discomfort for individuals and their sleeping partners, significant and even fatal medical consequences can and do occur as a result of these conditions.

These breathing difficulties can be caused by a number of different pressure points or resisters in the breathing process. Some of these resistors include the nose, the soft palate, the area behind the tongue, and the epiglottis. Any of these resistors can cause snoring, and certain individuals have multiple resistors simultaneously contributing to their detriment.

Snoring is caused by a variety of factors which can include smoking, obesity, alcohol consumption, lack of exercise, and aging. All of these can have a negative influence on the general muscle tone of the upper airway, leading to an obstructed airway.

Several existing treatments exist today for snoring and OSA. One of the first surgical procedures, still in use today, is uvulopalatopharygoplasty (UPPP). This procedure was introduced in 1961 and was first used in the United States in 1981. This procedure reduces snoring through the removal of vibratory tissues. See Fujitsu, S., et al., Surgical Correction of Anatomic Abnormalities in Obstructive Sleep Apnea Syndrome: uvulopalatopharygoplasty, Otolaryngol Head Neck Surg., Vol. 89, pp 923-934 (November/December 1981). Unfortunately, UPPP suffers several drawbacks including significant expense and numerous long-term complications such as velopharyngeal incompetence (VPI) and palatal dryness. Further, studies find that only 46% of patients report a significant reduction of snoring over time.

Since UPPP, several other medical procedures have been devised and attempted. These include laser-assisted uvuloplataplasty (LAUP) and radio frequency ablation (RFA), both of which have drawbacks. See Philip D. Littlefield and Eric A. Mair, Snoring surgery: Which one is best for you?, ENT-Ear Nose & Throat Journal, Volume 78, Number 11, Pages 861-870 (November 1999).

A well-known, non-surgical treatment is a CPAP (Continuous Positive Air Pressure) device, which pumps air into a face mask worn by an individual. As the individual inhales and exhales, this increased air pressure within the mask and their respiratory system counteracts the tendencies of the relaxed throat and mouth muscles to discourage airway closing. Unfortunately these devices suffer from not being terribly comfortable, portable, or convenient, plus the masks don't always fit well, resulting in pressure loss. This results in a compliance rate of less than 50%. See Servidio U.S. Pat. No. 5,117,819.

Another common mechanism to treat mild snoring involves expanding the nasal passages in a variety of ways. This includes adhesive devices that affix to the outside of the nose, such as a “Breathe-Rite” strip, as well as devices that are designed to fit inside the nostrils and expand them from the inside-out. See Rush U.S. Pat. No. 6,375,667 and Corsaro U.S. Pat. No. 5,727,543, respectively. These devices can irritate the skin on the outside of the nose or the inner lining of the mucosa of the nasal passageway.

Airway Orthotics (AO) are oral appliances that are provided in two main types: the mandibular advancement device (MAD) and the tongue retention device (TRD). See Thornton U.S. Pat. No. 5,427,117 and Wirt U.S. Pat. No. 4,196,724 respectively.

MAD devices move the mandible, or lower jaw, forward in controlled increments, affording the dilation of the airway. This also tightens the upper airway via anterior and superior movement of the hyoid bone, stretching the infrahyoid muscles attached to the upper airway. Unfortunately, advancing the mandible can cause tempromandibular joint disorder (TMJ) and unpredictable occlusal (bite) changes.

While the aforementioned devices and techniques are intended to treat upper airway instability such as snoring or OSA, they are useful to a fairly limited subset of people exhibiting these difficulties. There are therefore a vast number of patients whose airway disorder is believed to be treatable using the preferred embodiment described herein.

Finally, brief mention is made of the adhesive nasal respiratory devices described by Doshi U.S. Pat. No. 7,506,649. Adhesive nasal respiratory devices could benefit some embodiments of the invention described herein.

### SUMMARY

This device has two primary functions that serve the purpose of reducing or eliminating snoring and mild obstructive sleep apnea (OSA). The first function is to provide for easy inhalation of air of a substantial volume. The second function is to leverage the exhalation process to generate positive pressure in the throat and mouth. The creation of this pressure is a natural and obvious result of exhalation process when the exhale egress is constrained. This can be achieved by restricting the exhalation egress to flow through the nasal cavity. This can be further restricted by manually restricting the flow of air as it exits the nose.

The result of the slight restriction in the exhalation process is that positive airway pressure is generated, offsetting breathing difficulties encountered during sleep as the muscle tone in the upper airway relaxes. This is similar to continuous positive airway pressure (CPAP) devices with the obvious difference being that the pressure is naturally created by the process of exhaling through a restricted exit.

Unlike other devices in this field, the proposed embodiment does not constrain the tongue or impede swallowing. It does not require surgery, and can very easily be used or removed at any time. The preferred embodiments are intended to be both affordable and very straightforward to use.

Preferred embodiments might naturally leverage other established techniques for reducing snoring, such as the general benefits recognized by having the mouth open or by utilizing mandibular advancement mechanisms that extend the user's lower jaw forward. These mechanisms discourage snoring and OSA by providing additional room between the tongue, soft palette, and related areas of the upper airway and mouth, and by tightening the soft tissue and muscles of the upper airway.

These and other purposes, features, and characteristics of the present invention, as well as the methods of operation and functions of the related elements of structure will be more apparent upon consideration of the following detailed description, claims, and drawings, all of which form a part of this specification. It should be expressly understood that the drawings are for the purpose of illustration and description only and are not intended to define the limits of the invention. Nor are the size or scale of the drawings intended to reflect actual size, scale, or proportion.

### BRIEF DESCRIPTION OF THE DRAWINGS

FIG. 1 illustrates an isometric view of an anti-snoring device according to the teachings of the present invention;

FIG. 2 illustrates an isometric view of one embodiment of the present invention from a different perspective;

FIG. 3 illustrates an embodiment utilizing a nasal respiratory restrictor to limit nasal exhalation;

FIG. 4 illustrates an isometric view of the additional nasal respiratory restrictor.

### DETAILED DESCRIPTION OF THE INVENTION

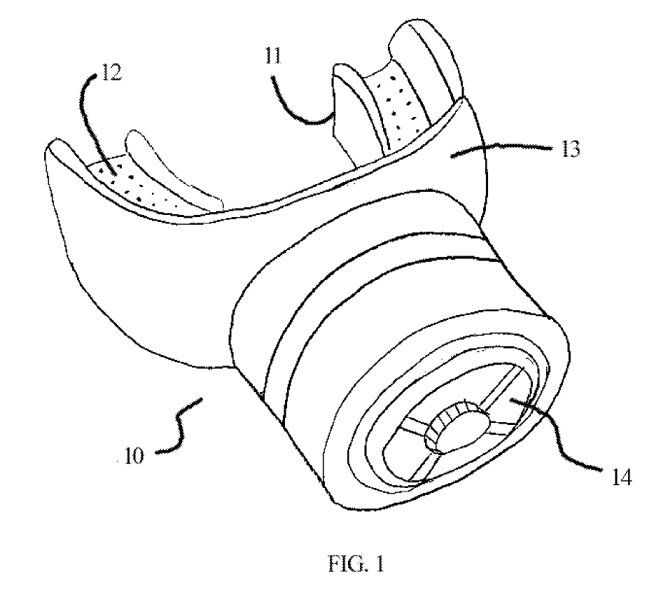

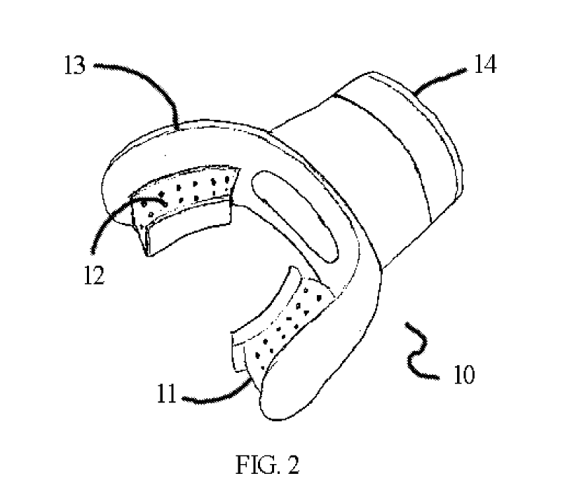

FIGS. 1-3 illustrate preferred embodiments of an anti-snoring device 10 according to the teachings of the present invention. As shown in FIG. 1, the anti-snoring device 10 is provided with a lower bite surface 11 and an upper bite surface 12. In one embodiment, the bite surface consists of silicon as one might find on a snorkel mouth piece. In other embodiments, the bite surface and other intraoral surfaces could be created from acrylic resin, various thermoplastics, ethylene vinyl acetate (EVA), or other common oral device materials such as those used for mandibular advancement purposes. An embodiment could, for example, be built in conjunction with a mandibular advancement device as described by Thornton et al. mentioned previously.

Bite surfaces 11 and 12 are used in connection with flange 13. 13 prevents the flow of air in or out of the mouth beyond where desired, through the adjustable, typically one-way, valve 14. In one embodiment, 13 may reside between the teeth and lips of a patient, effectively inside the mouth. This blocks air from entering or exiting the mouth other than through valve 14 or the nose, and it serves to help retain device 10 in the mouth during sleep. In another embodiment, 13 may reside outside of the mouth and bite surfaces 11 and 12 may be fitted to the teeth as with many mandibular advancement devices to help hold device 10 in the mouth.

Valve 14 in one embodiment can be a simple one-way purge valve with a silicon flap, such as one might find employed at the base of a snorkel tube. The role of valve 14 is typically to freely allow a substantial quantity of air into the mouth as desired by the patient, as quietly as possible. Valve 14 further is intended to restrict the egress of air from the mouth. Another embodiment would provide for the adjustment of the valve to allow customizable restricted oral exhalation and inhalation.

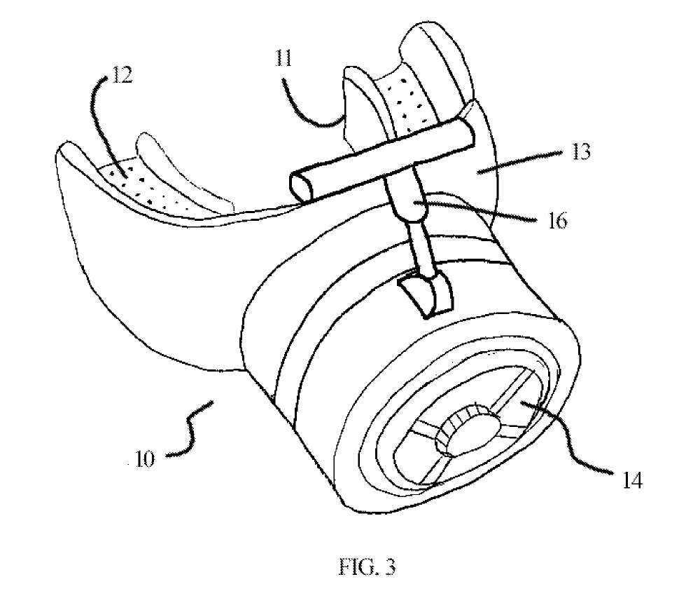

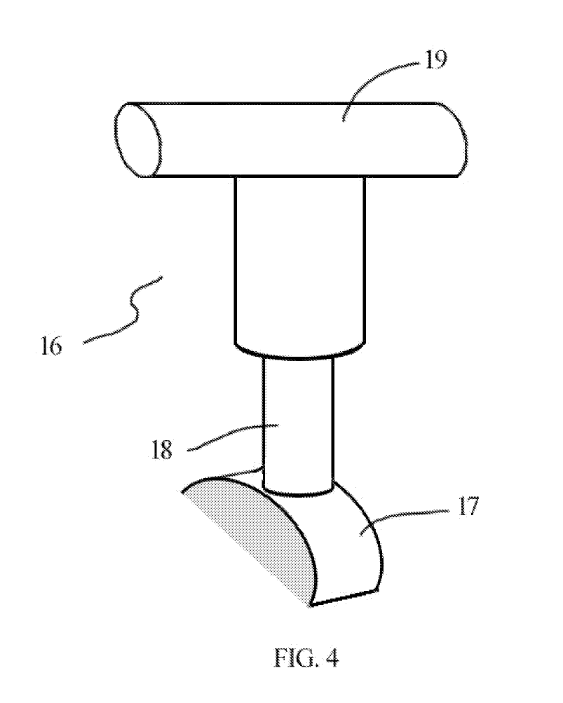

FIG. 3 illustrates an embodiment showing the usage of optional piece 16, illustrated in detail in FIG. 4.

FIG. 4 illustrates an optional and adjustable piece 16 intended to further restrict the egress of nasal exhalation. Adjustable piece 16 can flex toward or away from the nose on adjustable hinge 17. 16 can also be adjusted to be nearer to the nose by virtue of shortening or elongating the adjustable length shaft 18. 19 is a replaceable, soft cushion that rests near or touching the patient nose. 16 in general can quickly move to accommodate nose blowing. Other embodiments can be envisioned such as one that pushes the nostrils closed slightly or one that is not even attached to device 10. Further embodiments could provide a flexible connection that holds the nose restriction cushion generally in the same location for all users.

As a patient inhales, air enters the respiratory system. The lack of force required to draw air into the system is important, as a substantial vacuum in the upper airway would encourage blockages that cause snoring. In some sleepers, the inability to draw a sufficient amount of air in nasally results in an open-mouth snore that occurs upon inhalation.

As a patient exhales, air is intentionally constrained upon exiting the respiratory system. Valve 14 in its simplest form is a simple one-way flap valve, although alternate embodiments could provide a degree of adjustability in either direction. Since air cannot generally exit this valve, pressure is generated, effectively forcing many of the previously discussed snoring resistors apart. This pressure is sometimes referred to as positive end-expiratory pressure (PEEP) or expiratory positive airway pressure (EPAP). By providing positive pressure during exhalation, many exhalation snoring difficulties are abated or eliminated. Consequently, the closed circuit of breathing in through the mouth and out through the nose reduces or eliminates many causes of snoring.

Although the present invention and its advantages have been described in detail, it should be understood that various substitutions, alterations, or changes can be made herein without materially diverging from the scope and intent of the invention described herein.

What is claimed is:

1. An oral and nasal system that reduces snoring and obstructive sleep apnea (OSA) by allowing air inhalation in conjunction with restricted air exhalation.

2. An oral device of claim 1, wherein air is only allowed to enter the mouth, not exit the mouth.

3. An adjustable nasal device of claim 1, used in conjunction with the oral device of claim 2, wherein air can be restricted as it enters or exits the nose.

4. The method of applying positive airway pressure to the upper respiratory system including the throat, mouth, and nasal passages by restricting exhalation through the mouth and nose.

5. The method of claim 1 that generates positive airway pressure without a blower or pump.
